(19) United States
(12) Patent Application Publication
(10) Pub. No.: US 2008/0021464 A1
(43) Pub. Date: Jan. 24, 2008

(54) SYSTEM AND METHOD FOR A SPINAL IMPLANT LOCKING ASSEMBLY

(76) Inventors: Joshua Morin, Attleboro, MA (US); Arnold Oyola, Northborough, MA (US); Michael Perriello, Hopedale, MA (US)

Correspondence Address: CARR LLP (IST), 670 FOUNDERS SQUARE, 900 JACKSON STREET, DALLAS, TX, 75202, US

(21) Appl. No.: 11/780,343

(22) Filed: Jul. 19, 2007

Related U.S. Application Data
(60) Provisional application No. 60/831,879, filed on Jul. 19, 2006.

(51) Int. Cl. A61B 17/58; A61B 17/56

(52) U.S. Cl. 606/61; 606/73

(57) ABSTRACT

Provided are a system and method for a spinal implant locking assembly. In one example, the system includes a bone anchor, a polyaxial head coupled to a proximal end of the bone anchor, a spinal implant, and a locking assembly. The locking assembly may have a bearing post with a distal portion coupled to the polyaxial head and a proximal portion that extends through an opening in the spinal implant. The locking assembly may also have a bushing, a bearing element, and a threaded locking member. The threaded locking member may be configured to rotationally engage a threaded surface on at least one of the bearing post and the bushing.

### CLAIM OF PRIORITY

This application claims priority from U.S. Provisional Patent Application Ser. No. 60/831,879, filed on Jul. 19, 2006, and entitled “LOCKING ASSEMBLY”, which is incorporated herein in its entirety.

### BACKGROUND

The human spine is a complex structure designed to achieve a myriad of tasks, many of them of a complex kinematic nature. The spinal vertebrae allow the spine to flex in three axes of movement relative to the portion of the spine in motion. These axes include the horizontal (bending either forward/anterior or aft/posterior), roll (bending to either left or right side) and vertical (twisting of the shoulders relative to the pelvis).

In flexing about the horizontal axis, into flexion (bending forward or anterior) and extension (bending backward or posterior), vertebrae of the spine must rotate about the horizontal axis, to various degrees of rotation. The sum of all such movement about the horizontal axis of produces the overall flexion or extension of the spine. For example, the vertebrae that make up the lumbar region of the human spine move through roughly an arc of 15° relative to its adjacent or neighboring vertebrae. Vertebrae of other regions of the human spine (e.g., the thoracic and cervical regions) have different ranges of movement. Thus, if one were to view the posterior edge of a healthy vertebrae, one would observe that the edge moves through an arc of some degree (e.g., of about 15° in flexion and about 5° in extension if in the lumbar region) centered around a center of rotation. During such rotation, the anterior (front) edges of neighboring vertebrae move closer together, while the posterior edges move farther apart, compressing the anterior of the spine. Similarly, during extension, the posterior edges of neighboring vertebrae move closer together, while the anterior edges move farther apart, compressing the posterior of the spine. Also during flexion and extension, the vertebrae move in horizontal relationship to each other, providing up to 2-3 mm of translation.

In a normal spine, the vertebrae also permit right and left lateral bending. Accordingly, right lateral bending indicates the ability of the spine to bend over to the right by compressing the right portions of the spine and reducing the spacing between the right edges of associated vertebrae. Similarly, left lateral bending indicates the ability of the spine to bend over to the left by compressing the left portions of the spine and reducing the spacing between the left edges of associated vertebrae. The side of the spine opposite that portion compressed is expanded, increasing the spacing between the edges of vertebrae comprising that portion of the spine. For example, the vertebrae that make up the lumbar region of the human spine rotate about an axis of roll, moving through roughly an arc of 10° relative to its neighbor vertebrae, throughout right and left lateral bending.

Rotational movement about a vertical axis relative to the portion of the spine moving is also natural in the healthy spine. For example, rotational movement can be described as the clockwise or counter-clockwise twisting rotation of the vertebrae during a golf swing.

The inter-vertebral spacing (between neighboring vertebrae) in a healthy spine is maintained by a compressible and somewhat elastic disc. The disc serves to allow the spine to move about the various axes of rotation and through the various arcs and movements required for normal mobility. The elasticity of the disc maintains spacing between the vertebrae, allowing room or clearance for compression of neighboring vertebrae, during flexion and lateral bending of the spine. In addition, the disc allows relative rotation about the vertical axis of neighboring vertebrae, allowing twisting of the shoulders relative to the hips and pelvis. Clearance between neighboring vertebrae maintained by a healthy disc is also important to allow nerves from the spinal chord to extend out of the spine, between neighboring vertebrae, without being squeezed or impinged by the vertebrae.

In situations (based upon injury or otherwise) where a disc is not functioning properly, the inter-vertebral disc tends to compress, and in doing so pressure is exerted on nerves extending from the spinal cord by this reduced inter-vertebral spacing. Various other types of nerve problems may be experienced in the spine, such as exiting nerve root compression in the neural foramen, passing nerve root compression, and ennervated annulus (where nerves grow into a cracked/compromised annulus, causing pain every time the disc/annulus is compressed), as examples. Many medical procedures have been devised to alleviate such nerve compression and the pain that results from nerve pressure. Many of these procedures revolve around attempts to prevent the vertebrae from moving too close to each other thereby maintaining space for the nerves to exit without being impinged upon by movements of the spine.

In one such procedure, screws are embedded in adjacent vertebrae pedicles and rigid rods or plates are then secured between the screws. In such a situation, the pedicle screws (which are in effect extensions of the vertebrae) then press against the rigid spacer which serves to distract the degenerated disc space, maintaining adequate separation between the neighboring vertebrae, so as to prevent the vertebrae from compressing the nerves. This prevents nerve pressure due to extension of the spine; however, when the patient then tries to bend forward (putting the spine in flexion), the posterior portions of at least two vertebrae are effectively held together. Furthermore, the lateral bending or rotational movement between the affected vertebrae is significantly reduced, due to the rigid connection of the spacers. Overall movement of the spine is reduced as more vertebras are distracted by such rigid spacers. This type of spacer not only limits the patient's movements, but also places additional stress on other portions of the spine (typically, the stress placed on adjacent vertebrae without spacers being the worse), often leading to further complications at a later date.

In other procedures, dynamic fixation devices are used. However, conventional dynamic fixation devices do not facilitate lateral bending and rotational movement with respect to the fixated discs. This can cause further pressure on the neighboring discs during these types of movements, which over time, may cause additional problems in the neighboring discs.

What is needed are dynamic systems which approximate the motion of a healthy spine, yet provide for stabilization of a spine, and means for coupling such dynamic systems to a spine.

### SUMMARY

In one embodiment, a spinal implant system comprises a bone anchor, a polyaxial head coupled to a proximal end of the bone anchor, a spinal implant, and a locking assembly. The locking assembly has a bearing post, a bushing, a bearing element, and a threaded locking member. The bearing post includes a proximal portion and a distal portion coupled by a longitudinal axis, wherein the distal portion is coupled to the polyaxial head and the proximal portion extends through an opening in the spinal implant. The bushing has a first exterior surface and a first interior surface defining a first bore sized to receive the proximal portion of the bearing post, wherein the bushing is positioned on a first side of the opening. The bearing element is positioned on a second side of the opening and coupled to the bushing, and has a second exterior surface and a second interior surface defining a second bore sized to receive the proximal portion of the bearing post. The threaded locking member has a third exterior surface and a third interior surface defining a third bore sized to receive the proximal portion of the bearing post, wherein the threaded locking member is configured to rotationally engage a threaded surface on at least one of the proximal portion of the bearing post and the first interior surface of the bushing while enabling the bushing to rotate relative to the bearing post.

In another embodiment, a locking assembly for a spinal implant comprises a bearing post, a bushing, a bearing element, and a collet. The bearing post has a proximal portion and a distal portion coupled by a longitudinal axis, wherein the distal portion is configured to engage a polyaxial head. The bushing has a first exterior surface and a threaded first interior surface defining a first bore sized to receive the proximal portion of the bearing post. The bearing element has a second exterior surface and a second interior surface defining a second bore sized to receive the proximal portion of the bearing post, wherein the bearing element is coupled to the bushing. The collet has a threaded third exterior surface configured to rotationally engage the threaded first interior surface and a third interior surface defining a third bore sized to receive the proximal portion of the bearing post, wherein the collet is rotationally fixed relative to the bearing post and the bushing is not rotationally fixed relative to the bearing post.

In yet another embodiment, a locking assembly for a spinal implant comprises a bearing post, a bushing, a bearing element, and a locking cap. The bearing post has a threaded proximal portion and a distal portion coupled by a longitudinal axis, wherein the distal portion is configured to engage a polyaxial head. The bushing has a first exterior surface and a first interior surface defining a first bore sized to receive the proximal portion of the bearing post, wherein the first interior surface is threaded to engage the threaded proximal portion of the bearing post. The bearing element is coupled to the bushing and has a second exterior surface and a second interior surface defining a second bore sized to receive the threaded proximal portion of the bearing post. The locking cap has a third exterior surface and a threaded third interior surface defining a third bore sized to receive the proximal portion of the bearing post, wherein the threaded third interior surface is configured to rotationally engage the threaded proximal portion and wherein the bushing is configured to rotate relative to the bearing post.

In still another embodiment, a kit comprises a bone anchor, a polyaxial head configured to couple to a proximal end of the bone anchor, a bearing post, a spinal implant, and a threaded locking member. The bearing post has a proximal portion and a distal portion coupled by a longitudinal axis, wherein the distal portion includes a threaded surface configured to engage a threaded surface of the polyaxial head. The spinal implant includes an opening with a coupling means inserted therein, wherein the coupling means includes a first interior surface defining a first bore sized to slidingly receive the proximal portion and a first exterior surface facing a surface of the opening, and wherein the coupling means is configured to rotate relative to the spinal implant around an axis extending through the bore. The threaded locking member has a second exterior surface and a second interior surface defining a second bore sized to receive the proximal portion of the bearing post, wherein the threaded locking member is configured to rotationally engage a threaded surface on at least one of the proximal portion of the bearing post and the first interior surface.

In another embodiment, a method comprises inserting a bone anchor into a vertebral body, wherein the bone anchor is coupled to a polyaxial head, inserting a bearing post into the polyaxial head, adjusting a distance between a spinal implant and the polyaxial head by moving the spinal implant along a longitudinal axis of the bearing post, locking a position of the polyaxial head relative to the bone anchor using the bearing post, and securing a bushing coupled to the spinal implant to the bearing post by rotating the bushing relative to a locking member, wherein the rotation occurs around the longitudinal axis of the bearing post.

In yet another embodiment, a locking assembly comprises a bearing post, coupling means, and a locking member. The bearing post has a proximal portion and a distal portion coupled by a longitudinal axis. The coupling means is positioned in an opening formed through a spinal implant, wherein the coupling means includes a smooth exterior surface abutting a surface of the opening and a threaded interior surface defining a first bore configured to at least partially receive a locking member. The locking member has a threaded exterior surface and an interior surface defining a second bore sized to slidingly receive the proximal portion of the bearing post, wherein the threaded exterior surface is configured to rotationally engage the threaded interior surface.

### BRIEF DESCRIPTION OF THE DRAWINGS

Aspects of the present disclosure are best understood from the following detailed description when read with the accompanying figures. It is emphasized that various features may not be drawn to scale. In fact, the dimensions of various features may be arbitrarily increased or reduced for clarity of discussion.

FIG. 1 is an exploded view of one embodiment of a locking assembly.

FIG. 2 is a cross-sectional view of one embodiment of the locking assembly of FIG. 1 in an assembled state.

FIG. 3 is a perspective view of one embodiment of a locking assembly illustrated with a first dynamic stabilization device.

FIG. 4 is a perspective view of the first dynamic stabilization device of FIG. 3.

FIG. 5 is a more detailed view of one embodiment of the locking assembly and a portion of the first dynamic stabilization device of FIG. 3.

FIG. 6 is a perspective view of the locking assembly and dynamic stabilization device of FIG. 3 coupled to exemplary vertebrae.

FIG. 7 is a perspective view of one embodiment of a locking assembly illustrated with a second dynamic stabilization device.

FIG. 8 is a perspective view of the locking assembly and dynamic stabilization device of FIG. 7 coupled to exemplary vertebrae.

FIG. 9 is an exploded view of another embodiment of a locking assembly.

FIG. 10 is an assembled view of one embodiment of the locking assembly of FIG. 9.

FIG. 11 is a cross-sectional view of one embodiment of the locking assembly of FIG. 9.

FIG. 12 is a perspective view of one embodiment of a bearing post that may be used in the locking assembly of FIG. 9.

FIG. 13 is a perspective view of one embodiment of a lower surface of the bearing post of FIG. 12.

FIGS. 14A-14D illustrate various embodiments of the exterior surface of a proximal portion of the bearing post of FIG. 12.

FIG. 15 is a perspective view of one embodiment of a collet that may be used in the locking assembly of FIG. 9.

FIG. 16 is a top view illustrating interaction between the bearing post of FIG. 12 and the collet of FIG. 15.

FIG. 17 is a perspective view of another embodiment of a collet that may be used in the locking assembly of FIG. 9.

FIG. 18 is a top view illustrating interaction between the bearing post of FIG. 12 and the collet of FIG. 17.

FIGS. 19A-19C illustrate various embodiments of interior and exterior surfaces of the collets of FIGS. 15 and 17.

FIGS. 20 and 21 are side views illustrating how a bearing post and a collet of one embodiment of the locking assembly of FIG. 9 may interact to allow height adjustment of the collet relative to the bearing post.

FIG. 22 is a flow chart illustrating one embodiment of a method for using a locking assembly.

### DETAILED DESCRIPTION

It is to be understood that the following disclosure provides many different embodiments, or examples, for implementing different features of the disclosure. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

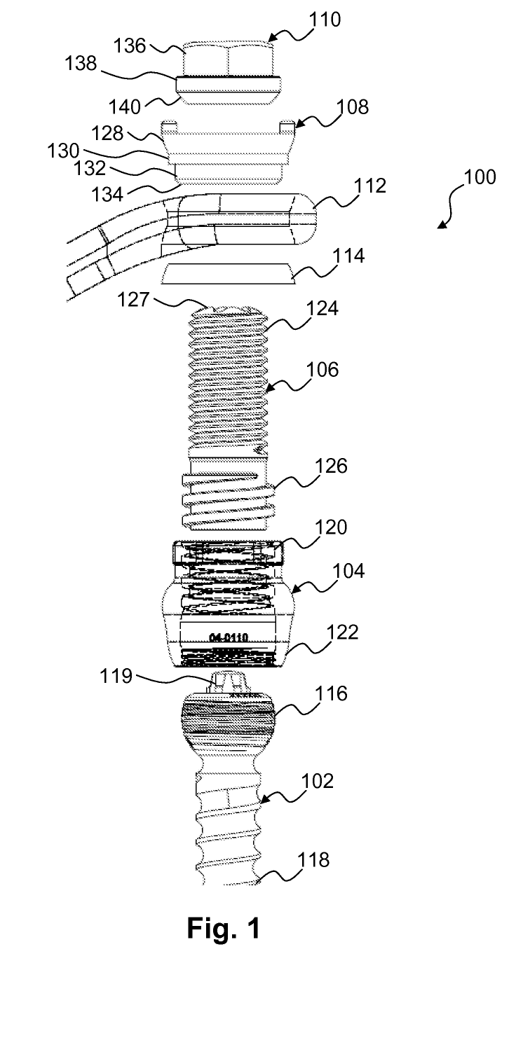

Referring to FIG. 1, in one embodiment, a locking assembly 100 is illustrated in an exploded view. The locking assembly 100 may include a bone anchor 102 (e.g., a pedicle screw), a polyaxial head 104, a bearing post 106, a threaded bushing 108, and a locking cap 110. The locking assembly 100 may be used to couple the bone anchor 102 to a connecting member 112 using, for example, a bearing element 114 coupled to the threaded bushing 108. Although not shown in FIG. 1, the connecting member 112 may be coupled to or part of a dynamic stabilization device used in stabilizing a portion of a spine while allowing at least a certain range of motion. The connecting member 112 may be part of the locking assembly 100 or may be considered to be separate from the locking assembly (e.g., may be part of the dynamic stabilization device). It is understood that the term “locking assembly” may refer to fewer parts than are illustrated in FIG. 1. For example, the locking assembly may not include the bone anchor 102, polyaxial head 104, or connecting member 112.

The bone anchor 102 may include a proximal portion 116 and a distal portion 118. In the present example, the proximal portion 116 may include a reverse thread that engages a compatible thread form within the polyaxial head 104. When coupled, the polyaxial head 104 may move in relation to the bone anchor 102. The bone anchor 102 may further include an engagement portion 119. Various examples of bone anchors and polyaxial heads are described in detail in U.S. patent application Ser. No. 10/690,211, filed on Oct. 21, 2003, U.S. patent application Ser. No. 10/990,272, filed on Nov. 16, 2004, and U.S. patent application Ser. No. 10/989,715, filed on Nov. 16, 2004, all of which are hereby incorporated by reference in their entirety.

The polyaxial head 104 may include a proximal portion 120 and a distal portion 122, both of which may be threaded. The proximal portion 120 may include a thread form different from that of the distal portion 122. In the present example, the distal portion 122 may be threaded to receive the reverse thread of the proximal portion 116 of the bone anchor 102. The proximal portion 120 may be threaded to receive a portion of the bearing post 106. The threads of the proximal portion 120 may be designed with anti-splay characteristics. For example, the threads may be grooved to accept a dovetail shaped thread. In some embodiments, the proximal portion 120 may be reverse threaded.

The bearing post 106 may include a proximal portion 124 and a distal portion 126, both of which may be threaded. The proximal portion 124 may include a thread form different from that of the distal portion 126. In the present example, the distal portion 126 may include a thread form configured to engage the thread form of the proximal portion 120 of the polyaxial head 104. Although the thread form is not reverse threaded in the present embodiment, it is understood that it may be reverse threaded in other embodiments. The proximal portion 124 may be threaded to engage the threaded bushing 108 and locking cap 110. The proximal end of the bearing post 106 may include one or more features 127. Such features 127 may, for example, be used to engage a tool for rotating the bearing post 106.

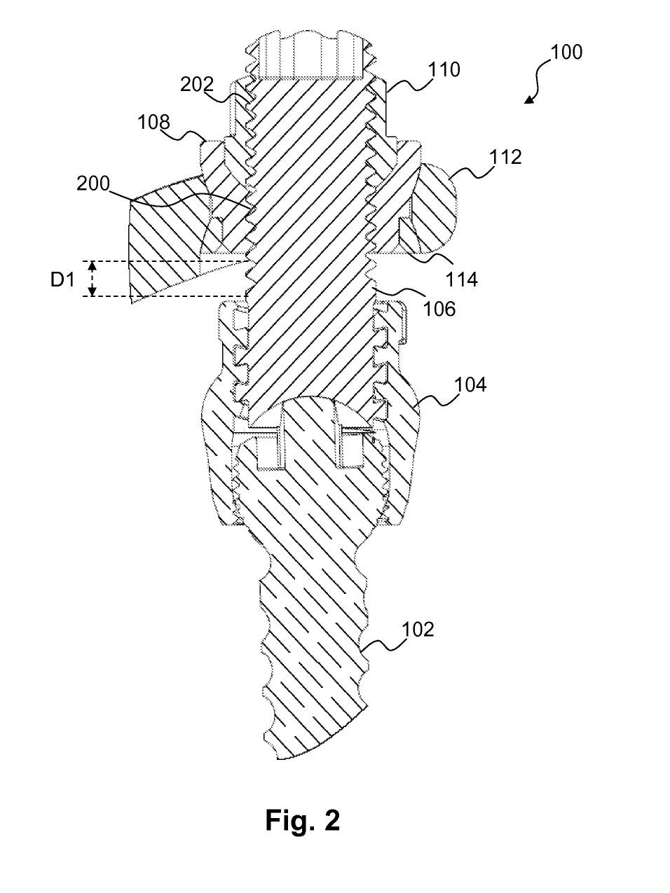

The threaded bushing 108 may include a threaded interior surface (e.g., reference number 200 of FIG. 2) configured to engage the proximal portion 124 of the bearing post 106. In the present example, the threaded bushing 108 may have an exterior surface of varying diameters, including a proximal portion 128, a first intermediate portion 130, a second intermediate portion 132, and a distal portion 134. As will be illustrated in FIG. 2, the distal portion 134 and second intermediate portion 132 may abut the bearing element 114 and the proximal portion 128 and first intermediate portion 130 may abut the connecting member 112. As the exterior surface of the threaded bushing 108 may be relatively smooth (e.g., non-threaded), the connecting member 112 may rotate around the threaded bearing element. In some embodiments, at least a portion of the exterior surfaces of the threaded bushing 108 and/or the bearing element 114 may act as a bearing surface against an inner surface of the connecting member 112. In such embodiments, one or more of the bearing surfaces may be polished and/or may be manufactured from materials with desirable bearing properties such as cobalt chrome.

The locking cap 110 may include a threaded interior surface (e.g., reference number 202 of FIG. 2) configured to engage the proximal portion 124 of the bearing post 106. In the present example, the locking cap 110 may have an exterior surface of varying diameters, including a proximal portion 136, an intermediate portion 138, and a distal portion 140. As will be illustrated in FIG. 2, the intermediate portion 138 and distal portion 140 may abut an interior surface of the threaded bushing 108 and the proximal portion 136 may provide a surface for engaging a tool used to tighten the locking cap 110.

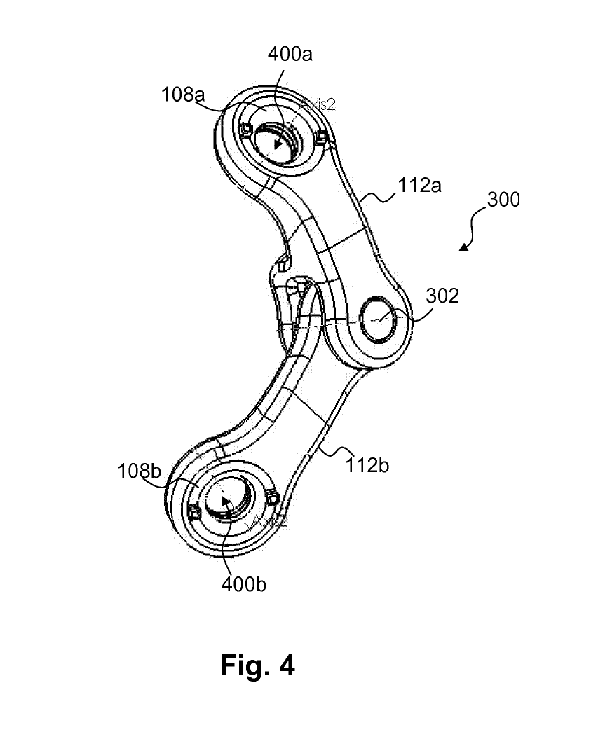

The connecting member 112 (e.g., a rod, slider, or a portion of a stabilization device) may include the threaded bushing 108 and/or the bearing element 114. For example, the bearing element 114 may be welded to the threaded bushing 108, thereby retaining both the bearing element and the threaded bushing in the connecting member 112. In the present embodiment, an opening (FIG. 4) in both the connecting member 112 and the bearing element 114 may be non-threaded to permit free rotation of the connecting member around the bearing post 106.

With additional reference to FIG. 2, one embodiment of the locking assembly 100 of FIG. 1 is illustrated in an assembled form. As stated previously, the polyaxial head 104 may generally move relative to the bone anchor 102. However, once the polyaxial head 104 is positioned as desired with respect to the bone anchor 102, it may be desirable to lock the polyaxial head into position. Accordingly, the bearing post 106 may be inserted into the polyaxial head 104 so that the threads of the distal portion 126 of the polyaxial locking member engage the threads of the proximal portion 120 of the polyaxial head. The bearing post 106 may then be tightened until the distal end (which may be concave in the present example) contacts the engagement portion 119. This locks the position of the polyaxial head 104 relative to the bone anchor 102.

As can be seen in FIG. 2, the threaded bushing 108 may not contact the polyaxial head 104. More specifically, using threaded surface 200, the position of the threaded bushing 108 may be adjusted along a longitudinal axis of the bearing post 106 to vary a distance “D1” that exists between the bearing element 114 and the polyaxial head 104. This enables a height of the connecting member 112 relative to the polyaxial head 104 to be varied and allows for adjustments to be made to a dynamic stabilization device coupled to the connecting member 112.

Using threaded surface 202, the locking cap 110 may be rotated along the longitudinal axis of the bearing post 106 to the desired position and tightened against the threaded bushing 108. As illustrated, intermediate portion 138 and distal portion 140 of the exterior surface of the locking cap 110 may enter a bore of the threaded bushing 108 (FIG. 4) and lock against an interior surface of the threaded bushing. This may lock the threaded bushing 108 into place relative to the polyaxial head 104 and may maintain the distance D1 as set.

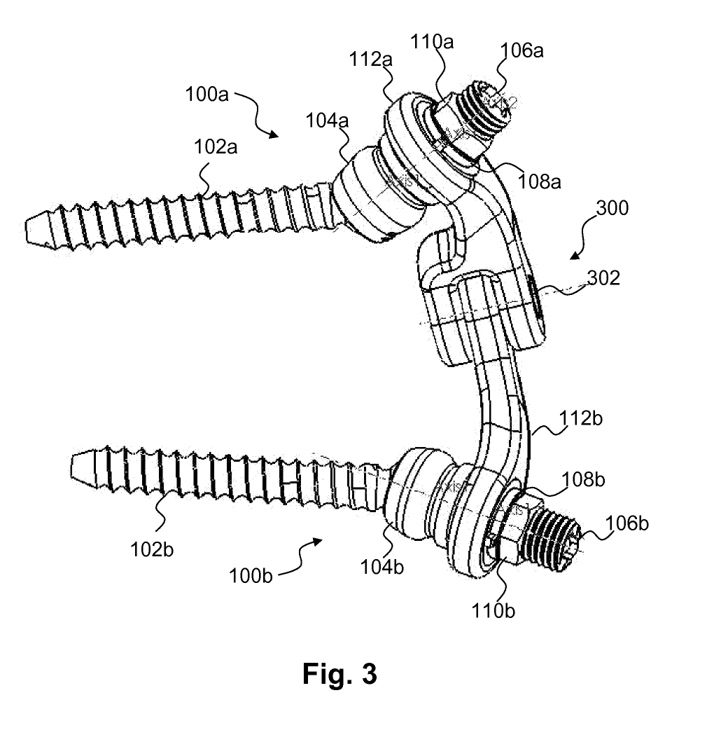

Referring to FIG. 3, in one embodiment, one or more locking assemblies 100a and 100b may be used with a spinal stabilization device 300. The locking assemblies 100a and 100b may be similar or identical to the locking assembly 100 of FIG. 1, and so like parts are referenced as in FIG. 1 except for the addition of an “a” or “b” suffix for purposes of convenience in distinguishing the two locking assemblies 100a and 100b. In the present example, the spinal stabilization device 300 is a dynamic stabilization device, such as that described in greater detail in U.S. Provisional Patent Application 60/656,126, filed on Feb. 5, 2005, and hereby incorporated by reference in its entirety. As the dynamic stabilization device 300 of FIGS. 3-6 is described more fully in the above incorporated patent application, it will not be discussed in detail herein except as an illustrative device with which the locking assembly 100 of FIG. 1 may be used.

The dynamic stabilization device 300 may include two connecting members 112a and 112b that are coupled via a pin 302. Although the connecting members 112a and 112b are part of the dynamic stabilization device 300 in the present example, it is understood that they may be separate from the dynamic stabilization device in other embodiments. It is understood that the two locking assemblies 100a and 100b may be separately adjusted with respect to the connecting members 112a and 112b, respectively. For example, the distance D1 (FIG. 2) may be different between the locking assemblies 100a and 100b. The angle at which each polyaxial head 104a and 104b is locked relative to the bone anchors 102a and 102b may also differ.

With additional reference to FIG. 4, one embodiment of the dynamic stabilization device 300 of FIG. 3 is illustrated with a portion of the locking assembly 100a. As illustrated in FIG. 4, each connecting member 112a and 112b may include a bore 400a and 400b, respectively, into which the threaded bushings 108a and 108b may be inserted. As described previously, an exterior surface of each of the threaded bushings 108a and 108b abutting the respective connecting members 112a and 112b may be relatively smooth to enable the connecting members to rotate around the threaded bushings.

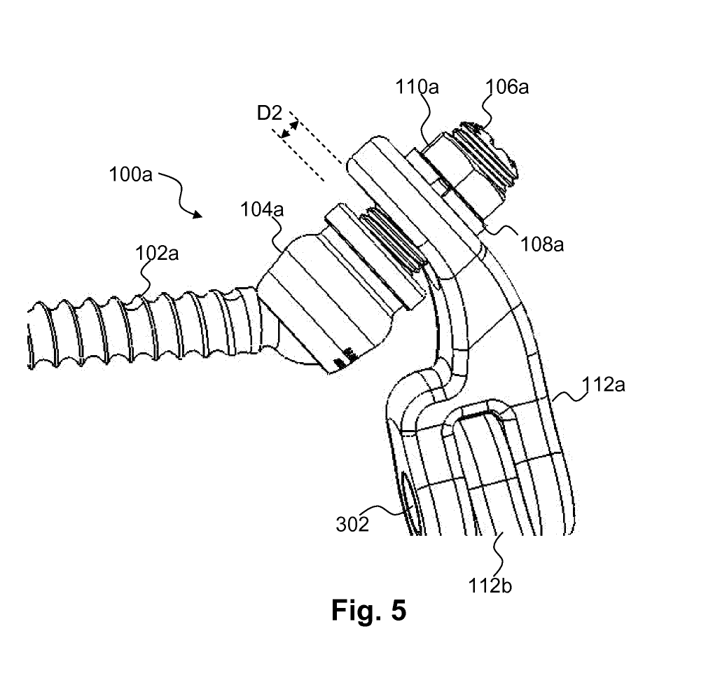

With additional reference to FIG. 5, a more detailed illustration of a portion of the locking assembly 100a and dynamic stabilization device 300 of FIG. 3 is provided. In the present embodiment, the connecting member 112a is separated from the polyaxial head 104a by a distance D2. The distance D2 may vary and, in some examples, may be zero if the connecting member 112a is in contact with the polyaxial head 104a. The locking cap 110a and threaded bushing 108a may be tightened to maintain the desired distance D2.

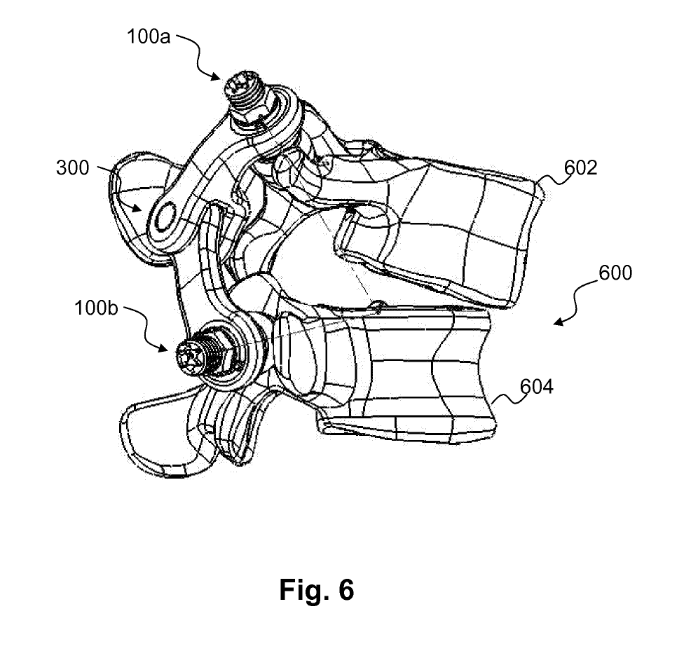

Referring to FIG. 6, one embodiment of the locking assemblies 100a, 100b, and dynamic stabilization device 300 of FIG. 3 is illustrated with a portion of a spine 600. More specifically, the locking assembly 100a is illustrated as coupling the dynamic stabilization device 300 to a vertebra 602 via the bone anchor 102a (FIG. 3). The locking assembly 100b is illustrated as coupling the dynamic stabilization device 300 to a vertebra 604 via the bone anchor 102b (FIG. 3). Although the locking assemblies 100a and 100b are illustrated as coupling the dynamic stabilization device 300 to the spine 600 in a vertical orientation, it is understood that other orientations and attachment points may be used.

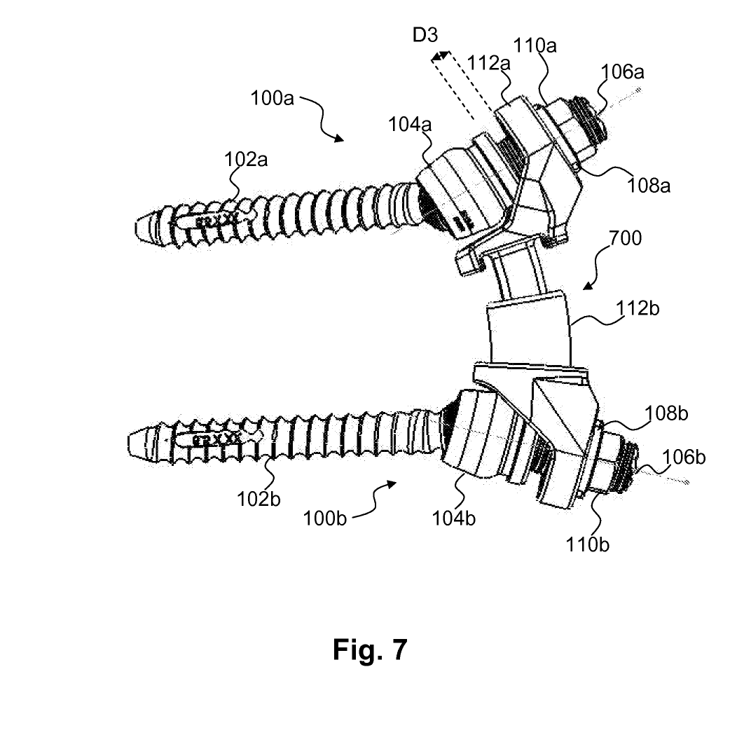

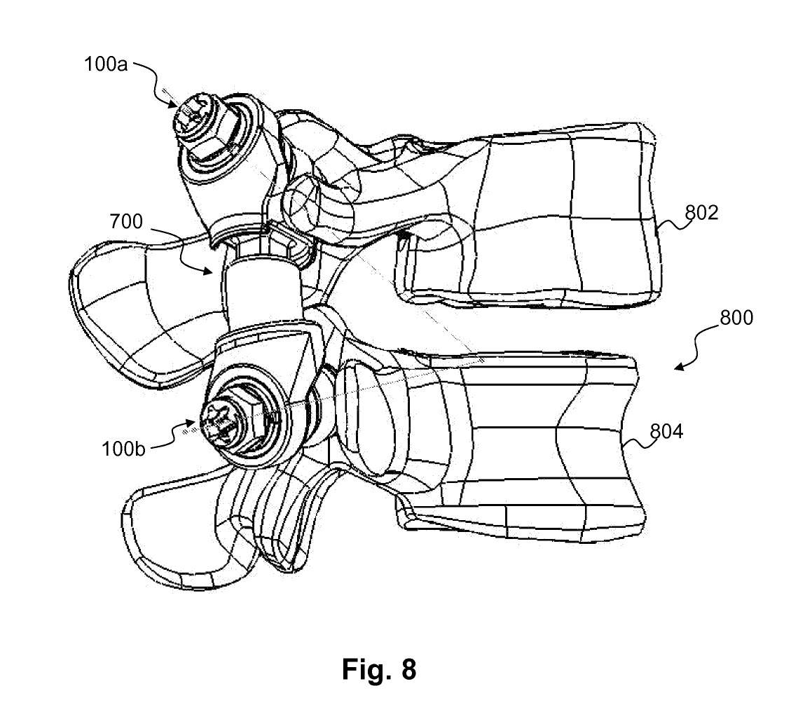

Referring to FIG. 7, in another embodiment, one or more locking assemblies 100a and 100b may be used with a spinal stabilization device 700. The locking assemblies 100a and 100b may be similar or identical to the locking assembly 100 of FIG. 1, and so like parts are referenced as in FIG. 1 except for the addition of an “a” or “b” suffix for purposes of convenience in distinguishing the two locking assemblies 100a and 100b. In the present example, the spinal stabilization device 700 is a dynamic stabilization device, such as that described in greater detail in U.S. Provisional Patent Application 60/637,324, filed on Dec. 16, 2004, and hereby incorporated by reference in its entirety. As the dynamic stabilization device 700 of FIGS. 7 and 8 is described more fully in the above incorporated patent application, it will not be discussed in detail herein except as an illustrative device with which the locking assembly 100 of FIG. 1 may be used.

The dynamic stabilization device 700 may include two members 112a and 112b that are coupled in a male/female relationship. Although the connecting members 112a and 112b are part of the dynamic stabilization device 700 in the present example, it is understood that they may be separate from the dynamic stabilization device in other embodiments. It is understood that the two locking assemblies 100a and 100b may be separately adjusted with respect to the connecting members 112a and 112b, respectively. For example, a distance D3 may be different between the locking assemblies 100a and 100b. The angle at which each polyaxial head 104a and 104b is locked relative to the bone anchors 102a and 102b may also differ.

Referring to FIG. 8, one embodiment of the locking assemblies 100a, 100b, and dynamic stabilization device 700 of FIG. 7 is illustrated with a portion of a spine 800. More specifically, the locking assembly 100a is illustrated as coupling the dynamic stabilization device 700 to a vertebra 802 via the bone anchor 102a (FIG. 7). The locking assembly 100b is illustrated as coupling the dynamic stabilization device 700 to a vertebra 804 via the bone anchor 102b (FIG. 7). Although the locking assemblies 100a and 100b are illustrated as coupling the dynamic stabilization device 700 to the spine 800 in a vertical orientation, it is understood that other orientations and attachment points may be used.

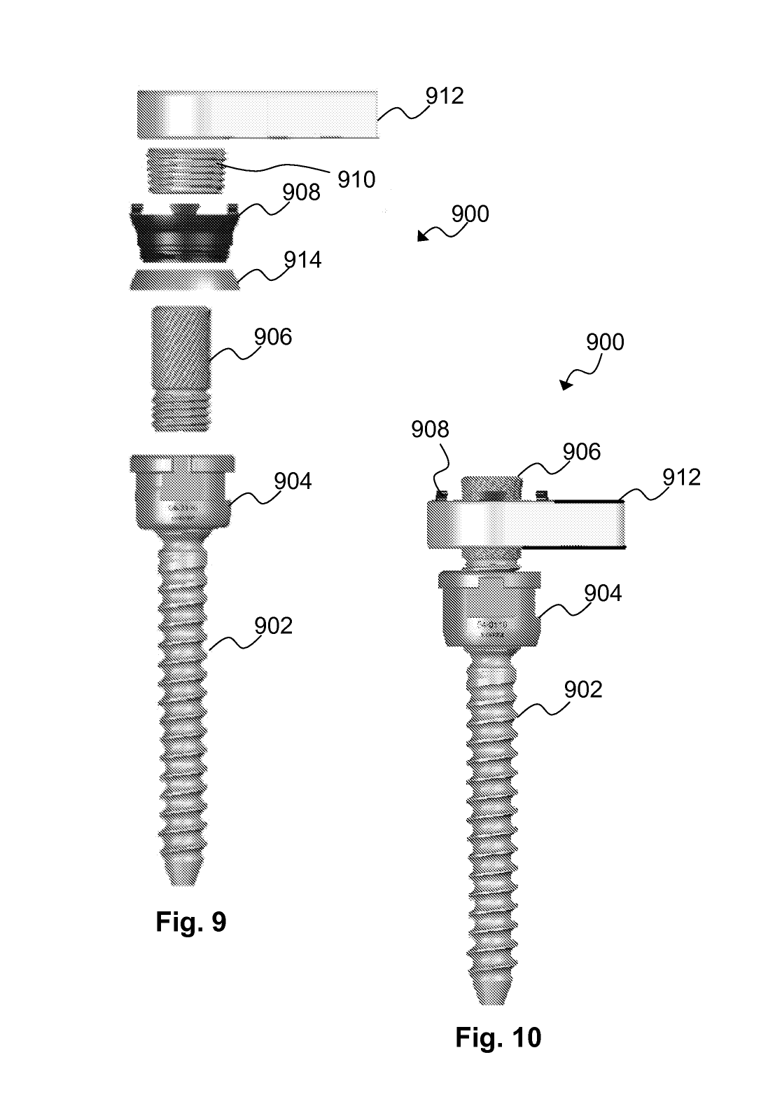

Referring to FIGS. 9 and 10, in another embodiment, a locking assembly 900 is illustrated in an exploded view and an assembled view, respectively. The locking assembly 900 may include a bone anchor 902 (e.g., a pedicle screw), a polyaxial head 904, a bearing post 906, a threaded bushing 908, and a collet 910. The locking assembly 900 may be used to couple the bone anchor 902 to a connecting member 912 using, for example, a bearing element 914 coupled to the threaded bushing 908. Although not shown in FIG. 9 or 10, the connecting member 912 may be coupled to or part of a dynamic stabilization device used in stabilizing a portion of a spine while allowing at least a certain range of motion.

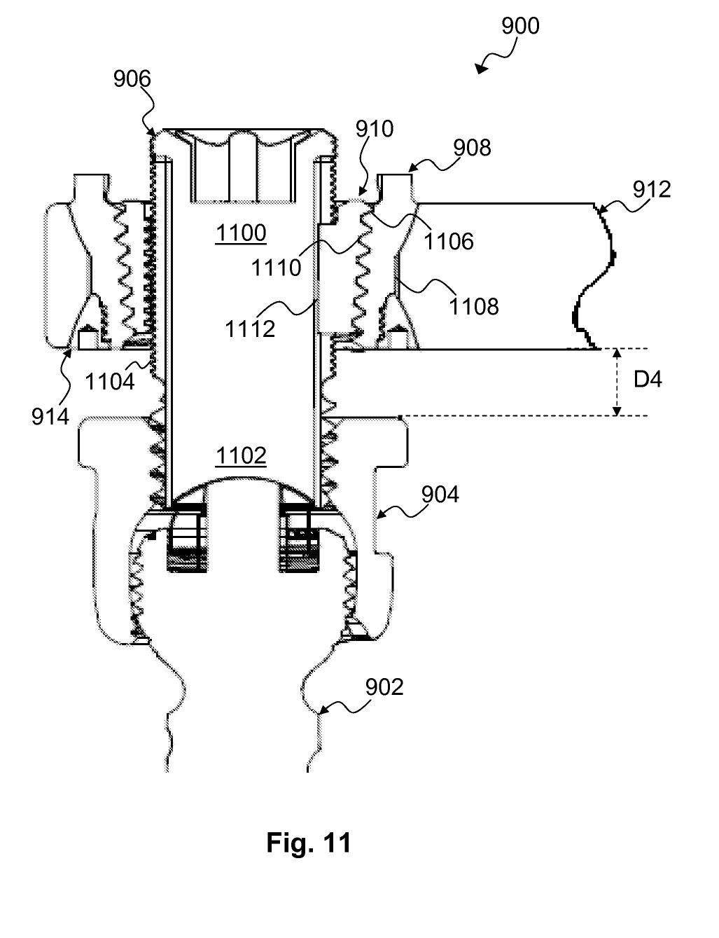

Referring to FIG. 11, a cross-sectional view of one embodiment of the locking assembly 900 of FIGS. 9 and 10 is illustrated. The bone anchor 902 and polyaxial head 904 may be similar or identical to the bone anchor 102 and polyaxial head 104 of FIG. 1 and are not described in detail in the present example.

In some embodiments, at least a portion of the exterior surfaces of the threaded bushing 908 and/or the bearing element 914 may act as a bearing surface against an inner surface of the connecting member 912. In such embodiments, one or more of the bearing surfaces may be polished and/or may be manufactured from materials with desirable bearing properties such as cobalt chrome.

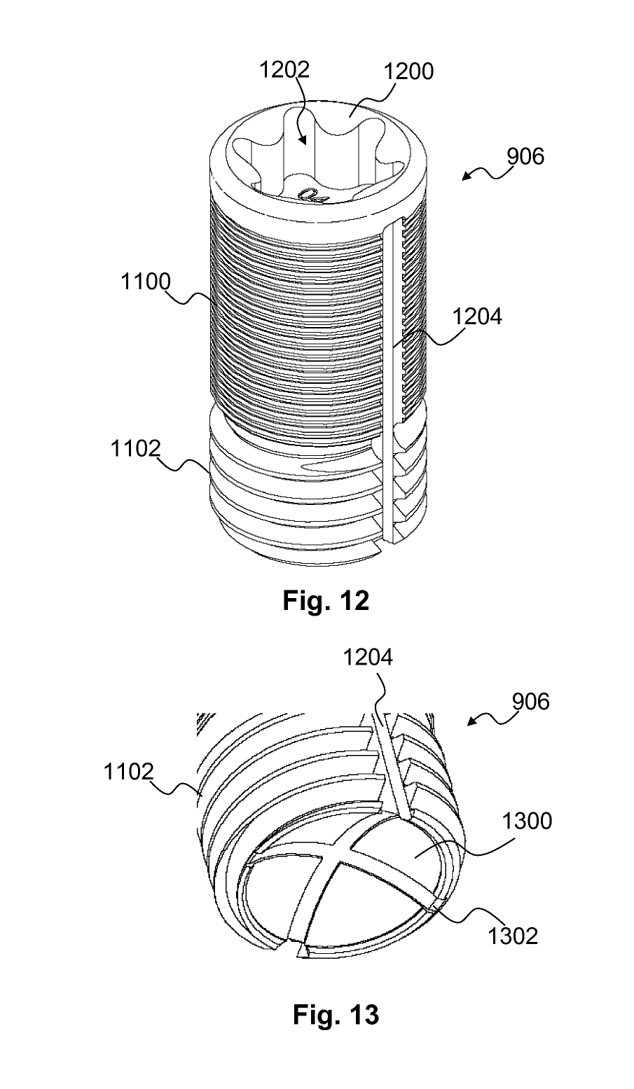

With additional reference to FIGS. 12 and 13, one embodiment of the bearing post 906 is illustrated in greater detail. The bearing post 906 may include a proximal portion 1100 and a distal portion 1102. In the present example, the distal portion 1102 may include a thread form configured to engage a thread form of a proximal portion of the polyaxial head 904. Although the thread form may not be reverse threaded in the present embodiment, it is understood that it may be reverse threaded in other embodiments.

As shown in FIG. 13, the end 1300 of the distal portion 1102 may be concave to receive the proximal portion of the bone anchor 902. In some embodiments, surface features 1302 (e.g., grooves) may be included to provide additional gripping of the bone anchor. The surface features 1302 may engage surface features of the bone anchor 902 or may provide additional engagement of the bone anchor even if the bone anchor lacks similar surface features.

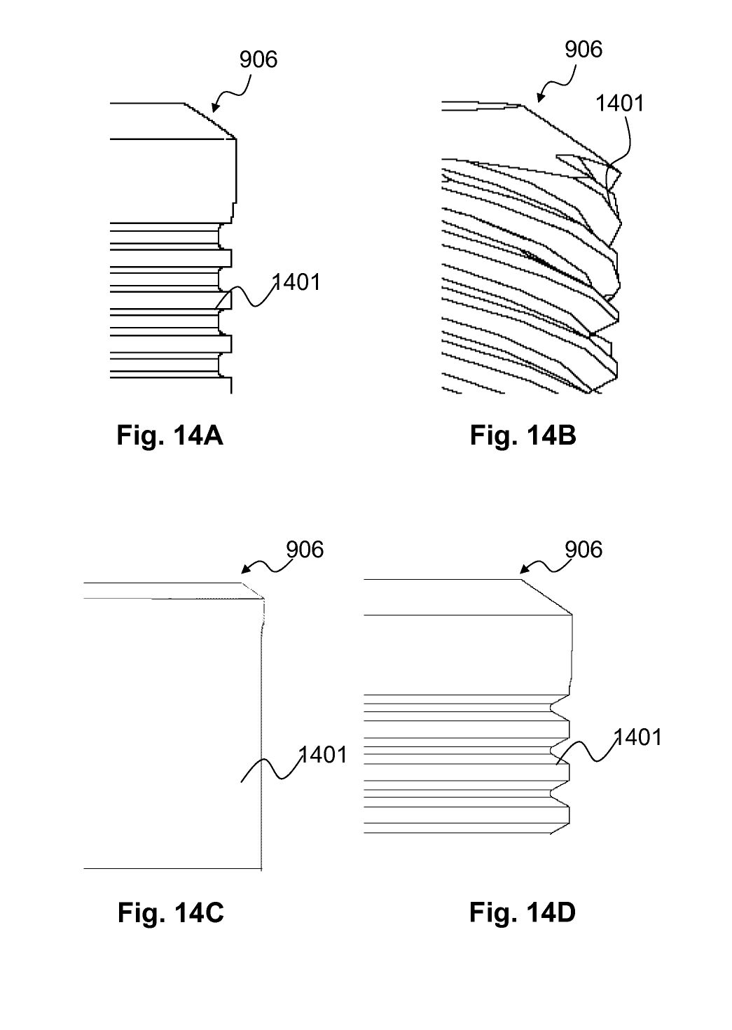

With additional reference to FIGS. 14A-14D, the proximal portion 1100 may be smooth or may have surface features 1401 (as shown in FIG. 12) configured to provide a gripping surface with respect to an interior surface of the collet 910. As illustrated in FIGS. 14A-14D, the surface features 1401 of the proximal portion 1100 may be in a variety of regular and/or irregular patterns. For example, regular patterns may be machined and irregular patterns may be created by treating the surface (e.g., bead blasting or creating a grit finish).

The proximal end 1200 of the bearing post 906 may include one or more features 1202. Such features 1202 may, for example, be used to engage a tool for rotating the bearing post 906. The bearing post 906 may also include a groove or other feature 1204 (FIG. 12) that may be used to align the collet 910 with the bearing post and/or to prevent rotation of the collet with respect to the bearing post. It is understood that the groove or other feature 1204 may not extend the entire length of the bearing post 906.

Referring again specifically to FIG. 9, the threaded bushing 908 may have an interior surface 1106 and an exterior surface 1108. The interior surface 1106 may be threaded to engage a threaded exterior surface 1110 of the collet 910. In the present example, the exterior surface 1108 of the threaded bushing 908 may have varying diameters, as described with respect to FIG. 1. The exterior surface 1108 of the threaded bushing 908 may be relatively smooth to enable the connecting member 912 to rotate around the threaded bushing and the bearing element 914.

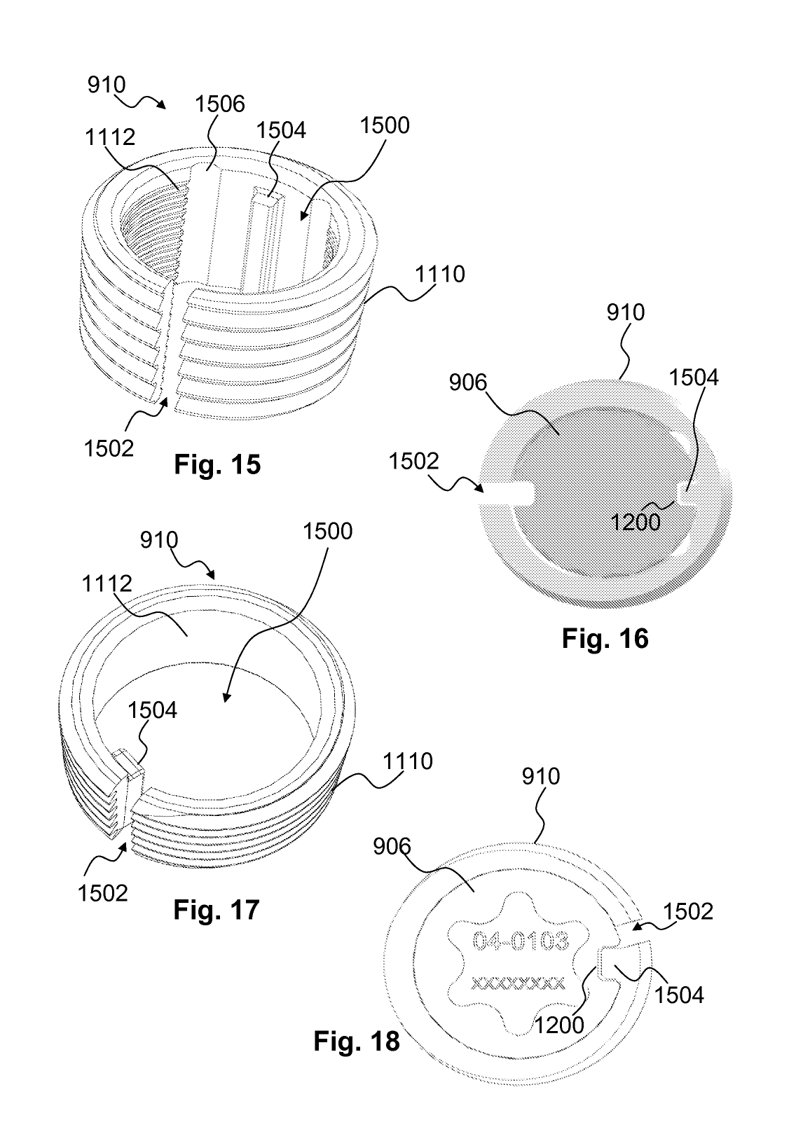

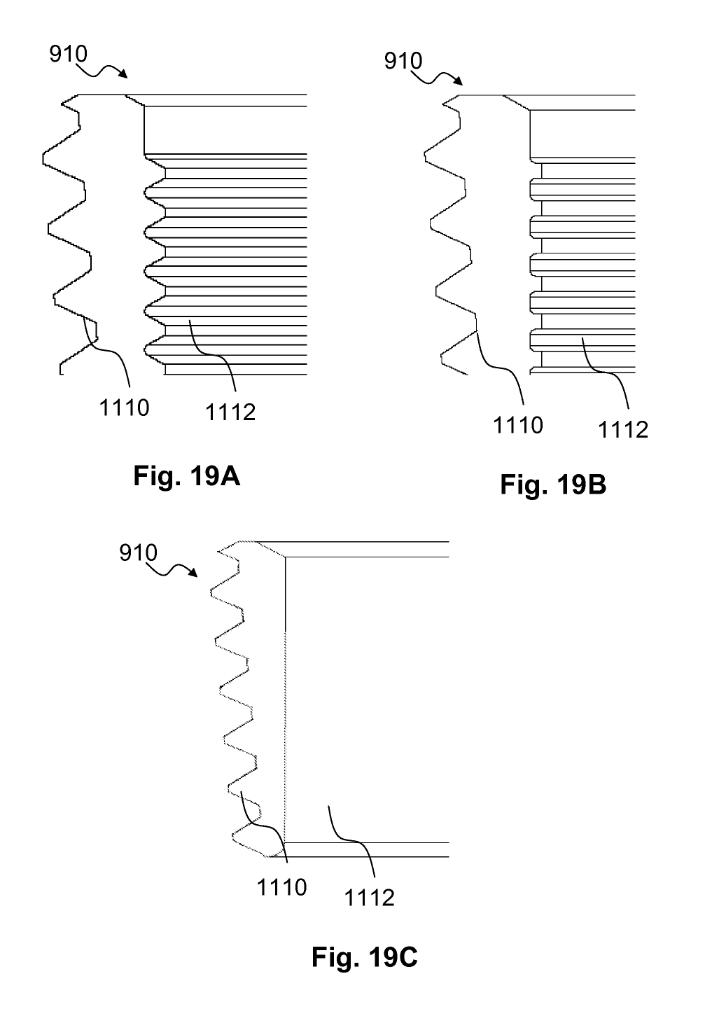

With continued reference to FIG. 9 and with additional reference to FIG. 15, one embodiment of the collet 910 is illustrated. In the present example, the collet 910 has an exterior surface 1110 that may be threaded to engage the interior threads 1106 of the threaded bushing 908. An interior surface 1112 of the collet 910 may have surface features or may be smooth. With additional reference to FIGS. 19A-19C, cross-sections of various embodiments of the collet 910 are illustrated. All or a portion of the interior surface 1112 may be smooth (FIG. 19C), or may be textured to have, for example, a grit finish. FIGS. 19A and 19B illustrate embodiments where the interior surface 1112 includes more substantial surface features. It is understood that many different processes may be used to create texturing or other features, including bead blasting, Electrical Discharge Machining (EDM), and/or other processes applied either during or after manufacture.

In the present example, the interior surface 1112 may define a bore 1500. A slot 1502 may be formed in the collet 910. The slot 1502 may enable the collet 910 to be compressed, thereby reducing the size of the bore 1500. The compression may aid in securing the collet 910 to the bearing post 906. A key 1504 or other surface feature may be present on the collet 910. The key 1504 may engage the groove 1204 (FIG. 12) of the bearing post 906 to prevent rotation of the collet 910 relative to the bearing post. Although the key 1504 is illustrated as a projection from the interior surface 1112, it is understood that the key may be a groove or any other surface feature that engages a corresponding feature of the bearing post 906. In some embodiments, the bore 1500 may be configured to slidingly receive the bearing post 906 (e.g., the bore 1500 may be relatively straight and may have a relatively uniform diameter), while the exterior surface 1110 and/or threads formed on the exterior surface may be tapered to match a correspondingly tapered surface and/or threads of the bushing 908.

In the present example, one or more grooves 1506 may be formed on the interior surface 1112 (or exterior surface 1110) of the collet 910. The grooves 1506, which result in a thinner wall between the interior surface 1112 and exterior surface 1110 and may accordingly enable that portion of the wall to flex more easily, and may enable the slot 1502 to be narrowed or closed using less pressure.

With additional reference to FIG. 16, the collet 910 of FIG. 15 is illustrated with the bearing post 906 of FIG. 12. The present example illustrates alignment of the key 1504 with the groove 1204. As stated previously, the key/groove interaction may prevent rotation of the collet 910 with respect to the bearing post 906.

With continued reference to FIG. 9 and with additional reference to FIG. 17, another embodiment of the collet 910 is illustrated. In the present example, the key 1504 of the collet 910 may be positioned near the slot 1502, rather than opposite the slot as illustrated in the example of FIG. 15. This may give the collet a “G” shape when viewed from above. It is understood that the interior surface 1112 and exterior surface 1110 may be similar or identical to the surfaces described with respect to FIG. 15.

With additional reference to FIG. 18, the collet 910 of FIG. 17 is illustrated with the bearing post 906 of FIG. 12. The present example illustrates alignment of the key 1504 with the groove 1204. As stated previously, the key/groove interaction may prevent rotation of the collet 910 with respect to the bearing post 906.

Referring again specifically to FIG. 9, the connecting member 912 (e.g., a rod, slider, or a portion of a stabilization device) may receive the bearing element 914. For example, the bearing element 914 may be welded to the threaded bushing 908. In the present embodiment, an opening in both the connecting member 912 and the bearing element 914 may be non-threaded to permit free rotation of the connecting member around the bearing post 906.

As illustrated in FIG. 11, a position of the connecting member 912 relative to the polyaxial head 904 may be adjusted. In the present example, the connecting member 912 is separated from the upper surface of the polyaxial head by a distance D4, which may be adjusted within a defined range. This is illustrated in greater detail with respect to FIGS. 18 and 19, discussed below.

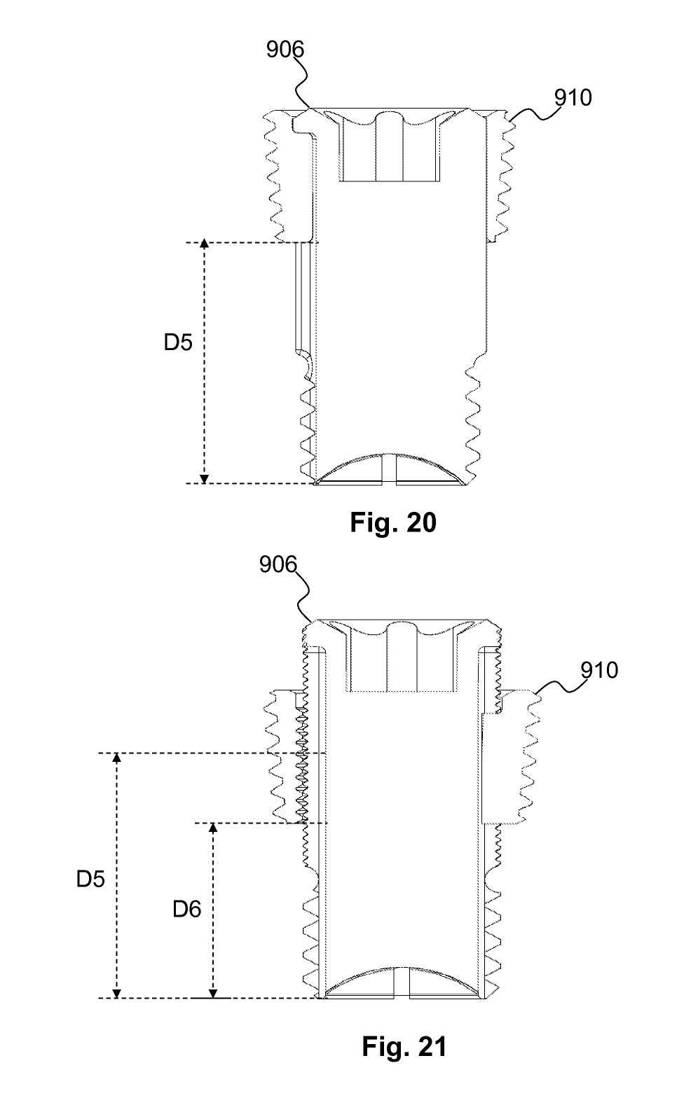

Referring to FIGS. 20 and 21, one embodiment of the bearing post 906 and collet 910 is illustrated in an assembled form. During assembly of the locking assembly, the bearing post 906 is inserted into the polyaxial head 904 and secured against the bone anchor 902. The position of the collet 910 may then be adjusted relative to the bearing post 906, which in turn adjusts the position of the connecting member 912 (FIG. 9) relative to the polyaxial head. In the present example, distances are described from the bottom surface of the collet 910 to the bottom surface of the bearing post 906.

In the example of FIG. 20, the collet 910 is illustrated in an uppermost position relative to the bearing post 906. A distance D5 separates the bottom surface of the collet 910 from the bottom surface of the bearing post 906. In the example of FIG. 21, the collet 910 is positioned closer to the distal end of the bearing post 906, with a distance D6 separating the bottom surface of the collet from the bottom surface of the bearing post. Although not shown, the collet 910 may be moved closer to the distal end of the bearing post 906 and may, in some embodiments, abut the polyaxial head 904 (not shown). Accordingly, the anchor assembly 900 of FIG. 9 enables height adjustment of the connecting member 912 relative to the polyaxial head 904.

The number of positions in the defined range of movement between the collet 910 and the bearing post 906 may be infinite. For example, use of a grit finish or the creation of an EDM texture on the interior surface of the collet 910 and/or exterior surface of the bearing post 906 may enable the connecting member 912 to adjust over an infinite number of positions to an appropriate height given the particular anatomy of a patient. Although not illustrated, the use of more structured engaging surfaces may provide a defined number of positions. Accordingly, the anchor assembly 900 may provide a great deal of flexibility due to the selection of varying lengths of bearing posts, as well as the selection of surfaces for the collet/bearing post interface. In some embodiments, the use of bearing post that has a non-threaded proximal portion (e.g., the bearing post 906 of FIG. 9) may enable the collet and/or bushing to center on the bearing post more easily than may occur with a threaded proximal portion.

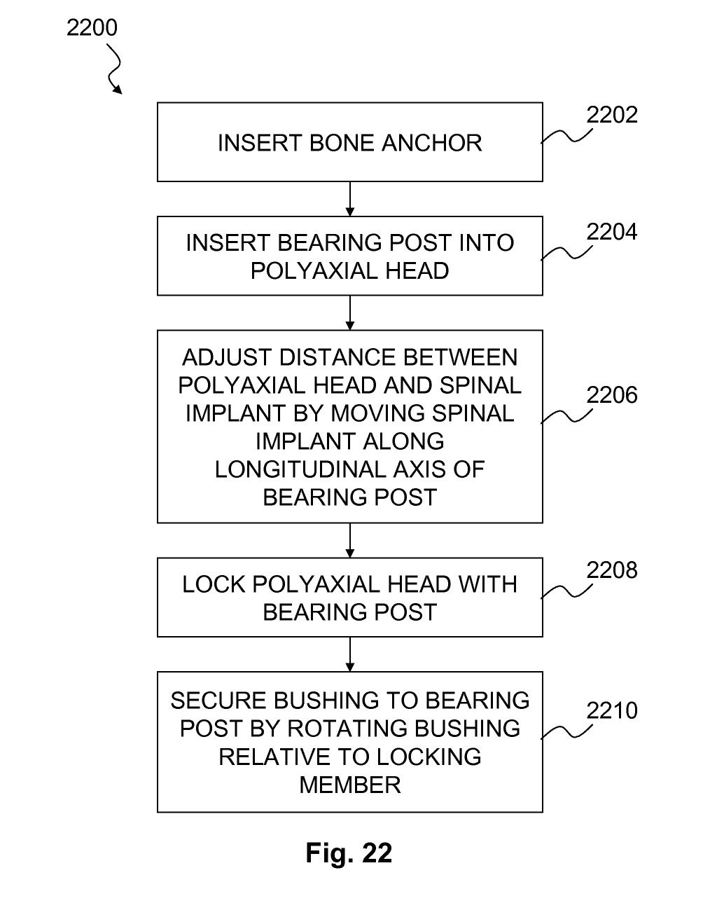

Referring to FIG. 22, one embodiment of a method 2200 is illustrated for using a locking assembly, such as one of the locking assemblies of FIG. 1 or FIG. 9. It is understood that steps may be included that are not described in detail in the present example, such as preparing a surgical site, making an incision in the patient, and inserting and removing dilators. As such steps are known to those of skill in the art, they are not included herein.

In step 2202, a bone anchor may be inserted into a vertebral body. In the present example, the bone anchor may include a polyaxial head that is coupled to a proximal end of the bone anchor. In other embodiments, the polyaxial head may be attached to the bone anchor after the bone anchor has been inserted.

In step 2204, a bearing post may be inserted into the polyaxial head. As described previously, the bearing post may be threaded to engage corresponding threads in the polyaxial head. In the present example, the bearing post may not be fully tightened in the present step, thereby allowing the polyaxial head to move relative to the bone anchor.

In step 2206, a distance may be adjusted between the spinal implant and the polyaxial head. The distance may be a predefined distance or the distance may be selected using other methods.

In step 2208, the bearing post may be tightened so as to engage the proximal end of the bone anchor. This may in turn lock the position of the polyaxial head relative to the bone anchor.

In step 2210, a bushing coupled to the spinal implant may be secured to the bearing post by rotating the bushing relative to a locking member. As described above, various embodiments of locking members may operate differently. For example, if the locking member is a locking cap, the locking member may be threaded to engage corresponding threads on the bearing post. With such a locking member, rotation of the locking member may include rotation around the bearing post relative to the bushing. In another example, if the locking member is a collet, the locking member may be threaded to engage corresponding threads on the bushing. With such a locking member, rotation of the locking member may include rotation relative to the bushing but the locking member may be unable to rotate around the bearing post. Accordingly, different locking members may perform differently to secure the spinal implant to the bearing post.

It is understood that the locking assemblies described herein and their equivalents may be used for a variety of purposes. For example, as illustrated in FIGS. 6 and 8, the locking assemblies may be used with dynamic stabilization devices. In such embodiments, the height of a dynamic stabilization device relative to a polyaxial head or other reference point may be selected and locked as described above, while the dynamic stabilization device is free to rotate around one or both bearing posts. In other embodiments, the ability to rotate may also be locked to provide, for example, a height adjustable fusion device. In still other embodiments, the locking assemblies described herein may be used with any type of spinal implant, whether or not such an implant is capable of dynamic stabilization. Accordingly, the locking assemblies described herein may be used to allow height adjustment and/or rotation, or may be configured to limit or eliminate the height adjustment and/or rotation functionality.

Although only a few exemplary embodiments of this disclosure have been described in detail above, those skilled in the art will readily appreciate that many modifications are possible in the exemplary embodiments without materially departing from the novel teachings and advantages of this disclosure. Also, features illustrated and discussed above with respect to some embodiments can be combined with features illustrated and discussed above with respect to other embodiments. Accordingly, all such modifications are intended to be included within the scope of this disclosure.

What is claimed is:

1. A spinal implant system comprising:
a bone anchor;
a polyaxial head coupled to a proximal end of the bone anchor;
a spinal implant; and
a locking assembly having:
a bearing post with a proximal portion and a distal portion coupled by a longitudinal axis, wherein the distal portion is coupled to the polyaxial head and the proximal portion extends through an opening in the spinal implant;
a bushing having a first exterior surface and a first interior surface defining a first bore sized to receive the proximal portion of the bearing post, wherein the bushing is positioned on a first side of the opening;
a bearing element positioned on a second side of the opening and coupled to the bushing, the bearing element having a second exterior surface and a second interior surface defining a second bore sized to receive the proximal portion of the bearing post; and
a threaded locking member having a third exterior surface and a third interior surface defining a third bore sized to receive the proximal portion of the bearing post, wherein the threaded locking member is configured to rotationally engage a threaded surface on at least one of the proximal portion of the bearing post and the first interior surface of the bushing while enabling the bushing to rotate relative to the bearing post.

2. The spinal implant system of claim 1 wherein the threaded locking member is a collet, wherein the third exterior surface is threaded to engage threads positioned on the first interior surface of the bushing.

3. The spinal implant system of claim 2 wherein the bearing post includes a first surface feature extending along at least a portion of the longitudinal axis and wherein the collet includes an opposing second surface feature configured to mate with the first surface feature to prevent rotation of the collet relative to the bearing post.

4. The spinal implant system of claim 4 wherein the collet includes a slot extending in the direction of the longitudinal axis.

5. The spinal implant system of claim 2 wherein at least one of the third interior surface and the proximal portion of the bearing post includes one or more surface features configured to create a gripping interface between the collet and the bearing post.

6. The spinal implant system of claim 1 wherein the threaded locking member is a locking cap, wherein the third interior surface is threaded to engage threads positioned on the proximal portion of the bearing post.

7. The spinal implant system of claim 1 wherein a distance between the polyaxial head and the spinal implant is adjustable.

8. A locking assembly for a spinal implant comprising:
a bearing post having a proximal portion and a distal portion coupled by a longitudinal axis, wherein the distal portion is configured to engage a polyaxial head;
a bushing having a first exterior surface and a threaded first interior surface defining a first bore sized to receive the proximal portion of the bearing post;
a bearing element having a second exterior surface and a second interior surface defining a second bore sized to receive the proximal portion of the bearing post, wherein the bearing element is coupled to the bushing; and
a collet having a threaded third exterior surface configured to rotationally engage the threaded first interior surface and a third interior surface defining a third bore sized to receive the proximal portion of the bearing post, wherein the collet is rotationally fixed relative to the bearing post and the bushing is not rotationally fixed relative to the bearing post.

9. The locking assembly of claim 8 wherein the bearing post includes a first surface feature extending along at least a portion of the longitudinal axis and wherein the collet includes an opposing second surface feature on the third interior surface configured to mate with the first surface feature to prevent rotation of the collet relative to the bearing post.

10. The locking assembly of claim 8 wherein the collet includes a slot extending in the direction of the longitudinal axis.

11. The locking assembly of claim 10 wherein the second surface feature is positioned near the slot.

12. The locking assembly of claim 10 wherein the second surface feature is positioned opposite the slot.

13. The locking assembly of claim 8 wherein at least one of the third interior surface and the proximal portion includes a plurality of surface features.

14. The locking assembly of claim 13 wherein the at least one of the third interior surface and the proximal portion are bead blasted.

15. The locking assembly of claim 13 wherein the at least one of the third interior surface and the proximal portion are grit finished.

16. The locking assembly of claim 13 wherein the at least one of the third interior surface and the proximal portion are machined to form substantially geometric surface features.

17. The locking assembly of claim 8 wherein the third interior surface includes at least one groove running substantially in the direction of the longitudinal axis.

18. The locking assembly of claim 8 wherein a position of the bushing along the longitudinal axis is adjustable.

19. The locking assembly of claim 8 wherein a lower surface of the bearing post is concave.

20. The locking assembly of claim 19 wherein the lower surface includes at least one surface feature configured to engage a bone anchor coupled to the polyaxial head.

21. A locking assembly for a spinal implant comprising:
a bearing post having a threaded proximal portion and a distal portion coupled by a longitudinal axis, wherein the distal portion is configured to engage a polyaxial head;
a bushing having a first exterior surface and a first interior surface defining a first bore sized to receive the proximal portion of the bearing post, wherein the first interior surface is threaded to engage the threaded proximal portion of the bearing post;
a bearing element coupled to the bushing and having a second exterior surface and a second interior surface defining a second bore sized to receive the threaded proximal portion of the bearing post; and
a locking cap having a third exterior surface and a threaded third interior surface defining a third bore sized to receive the proximal portion of the bearing post, wherein the threaded third interior surface is configured to rotationally engage the threaded proximal portion and wherein the bushing is configured to rotate relative to the bearing post.

22. The locking assembly of claim 21 wherein the bushing includes an indentation for at least partially receiving the locking cap.

23. The locking assembly of claim 21 wherein a lower surface of the bearing post is concave.

24. The locking assembly of claim 21 wherein a position of the bushing along the longitudinal axis is adjustable.

25. A kit comprising:
a bone anchor;
a polyaxial head configured to couple to a proximal end of the bone anchor;
a bearing post with a proximal portion and a distal portion coupled by a longitudinal axis, wherein the distal portion includes a threaded surface configured to engage a threaded surface of the polyaxial head;
a spinal implant including an opening with a coupling means inserted therein, wherein the coupling means includes a first interior surface defining a first bore sized to slidingly receive the proximal portion and a first exterior surface facing a surface of the opening, and wherein the coupling means is configured to rotate relative to the spinal implant around an axis extending through the bore; and
a threaded locking member having a second exterior surface and a second interior surface defining a second bore sized to receive the proximal portion of the bearing post, wherein the threaded locking member is configured to rotationally engage a threaded surface on at least one of the proximal portion of the bearing post and the first interior surface.

26. The kit of claim 25 wherein the coupling means includes a bushing having the first exterior surface and the first interior surface defining the first bore, wherein the bushing is positioned on a first side of the opening in the spinal implant, and a bearing element positioned on a second side of the opening and coupled to the bushing.

27. The kit of claim 25 wherein the threaded locking member is a collet, wherein the third exterior surface is threaded to engage threads positioned on the first interior surface of the bushing.

28. The kit of claim 25 wherein the threaded locking member is a locking cap, wherein the third interior surface is threaded to engage threads positioned on the proximal portion of the bearing post.

29. A method comprising:
inserting a bone anchor into a vertebral body, wherein the bone anchor is coupled to a polyaxial head;
inserting a bearing post into the polyaxial head;
adjusting a distance between a spinal implant and the polyaxial head by moving the spinal implant along a longitudinal axis of the bearing post;
locking a position of the polyaxial head relative to the bone anchor using the bearing post; and
securing a bushing coupled to the spinal implant to the bearing post by rotating the bushing relative to a locking member, wherein the rotation occurs around the longitudinal axis of the bearing post.

30. The method of claim 29 wherein securing the bushing coupled to the spinal implant to the bearing post includes rotating the locking member while holding the threaded bushing in a fixed position.

31. The method of claim 29 wherein securing the bushing coupled to the spinal implant to the bearing post includes rotating the threaded bushing while holding the locking member in a fixed position.

32. The method of claim 31 further comprising aligning a surface feature of the locking member with a corresponding surface feature of the bearing post to prevent rotation of the locking member relative to the bearing post.

33. A locking assembly comprising:
a bearing post with a proximal portion and a distal portion coupled by a longitudinal axis;
coupling means positioned in an opening formed through a spinal implant, wherein the coupling means includes a smooth exterior surface abutting a surface of the opening and a threaded interior surface defining a first bore configured to at least partially receive a locking member; and
the locking member having a threaded exterior surface and an interior surface defining a second bore sized to slidingly receive the proximal portion of the bearing post, wherein the threaded exterior surface is configured to rotationally engage the threaded interior surface.

34. The locking assembly of claim 33 wherein the locking member includes a first feature configured to engage a corresponding second feature of the bearing post.

35. The locking assembly of claim 33 wherein the coupling means includes a bushing having the smooth exterior surface and the first threaded surface defining the first bore, wherein the bushing is positioned on a first side of the opening in the spinal implant, and a bearing element positioned on a second side of the opening and coupled to the bushing.

36. The locking assembly of claim 33 wherein a thread form of the threaded exterior surface is tapered to match a taper of a thread form of the threaded interior surface.

37. The locking assembly of claim 33 wherein the threaded exterior surface is tapered to match a taper of the threaded interior surface.
